(19) United States
(12) Patent Application Publication
(10) Pub. No.: US 2023/0102536 A1
(43) Pub. Date: Mar. 30, 2023

(54) METHOD FOR DETERMINING A GAS CONCENTRATION AND GAS CONCENTRATION SENSOR

(71) Applicant: Infineon Technologies AG, Neubiberg (DE)

(72) Inventor: Christoph STEINER, St. Margarethen (AT)

(21) Appl. No.: 17/934,670

(22) Filed: Sep. 23, 2022

(30) Foreign Application Priority Data
Sep. 29, 2021 (DE) 102021125294.6

(51) Int. Cl. G01N 33/00; G01N 27/04

(52) U.S. Cl. CPC G01N 33/0062; G01N 27/04; G01N 33/005

(57) ABSTRACT

A method for determining a gas concentration in a cavity including: exciting a resistance sensor element situated in the cavity with an input signal, measuring an output signal of the resistance sensor element, determining a first parameter of a transfer function based on the input signal and the output signal, determining a second parameter of the transfer function based on the input signal and the output signal, checking a plausibility of the first parameter based on the second parameter, and outputting an error signal in the case of lack of plausibility of the first parameter.

### CROSS REFERENCE TO RELATED APPLICATION

This application claims priority to German Patent Application No. 102021125294.6 filed on Sep. 29, 2021, the content of which is incorporated by reference herein in its entirety.

### TECHNICAL FIELD

The present implementation relates to a method for determining a gas concentration and to a gas concentration sensor.

### BACKGROUND

Fossil energy carriers are increasingly being replaced by environmentally friendly fuels, also referred to as “green fuel”. By way of example, hydrogen generated with the aid of wind power installations, which is also referred to as green hydrogen, is used as an energy carrier for the drive of motor vehicles. In this context, sensors are required in order to detect the unwanted escape of hydrogen that can lead to the production of oxyhydrogen.

DE 10 2017 215527 A1 discloses a gas sensor for measuring a concentration of an analysis gas based on a thermal conductivity principle, comprising at least one analysis heating element arranged on a first membrane for heating the analysis gas, comprising a reference heating element arranged on a second membrane for heating a reference gas, comprising at least one evaluation electronics unit for measuring a change in resistance of the analysis heating element relative to an electrical resistance of the reference heating element, the change in resistance being caused by the analysis gas, wherein the first membrane and the second membrane are arranged adjacent to one another in a sensor substrate and wherein, by virtue of a base substrate arranged on one side on the sensor substrate, a measurement volume is able to be formed between the first membrane and the base substrate and a reference volume is able to be formed between the second membrane and the base substrate.

### SUMMARY

There is a need for a method for determining a gas concentration and a gas concentration sensor with higher operational safety.

Taking this as a departure point, a method and a gas concentration sensor as claimed in the independent patent claims are proposed. Advantageous configurations are specified in the dependent patent claims.

What is proposed is a method for determining a gas concentration in a cavity including: exciting a resistance sensor element situated in the cavity with an input signal, measuring an output signal of the resistance sensor element, determining a first parameter of a transfer function based on the input signal and the output signal, determining a second parameter of the transfer function based on the input signal and the output signal, checking a plausibility of the first parameter based on the second parameter, and outputting an error signal in the case of lack of plausibility of the first parameter.

What is likewise disclosed is a gas concentration sensor including a cavity for receiving a gas, a resistance sensor element arranged in the cavity, an excitation unit for exciting the resistance sensor element with an input signal, a measuring unit for determining an output signal of the resistance sensor element, an evaluation unit, wherein the evaluation unit is configured for carrying out the method.

### BRIEF DESCRIPTION OF THE DRAWINGS

Examples of the method and of the gas concentration sensor will now be explained in greater detail with reference to the figures, in which

FIG. 1 illustrates a gas concentration sensor;

FIG. 2 illustrates a model of a thermal system;

FIG. 3 illustrates parameter ranges of a transfer function; and

FIG. 4 illustrates parameter ranges of a transfer function.

### DETAILED DESCRIPTION

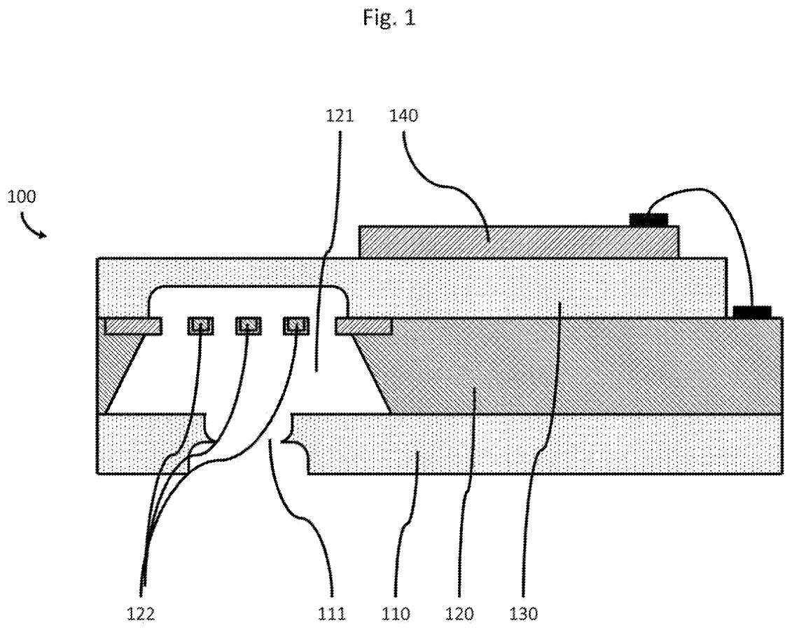

The gas concentration sensor 100 illustrated in FIG. 1 has a cavity 121, in which three resistance sensor elements 122 are arranged. The resistance sensor elements 122 can be produced from a processed semiconductor substrate 120. The cavity 121 can be delimited by a first cover 110 and a second cover 130. The first cover 110 and/or the second cover 130 can be glass elements, which are connected to the semiconductor substrate 120 by bonding.

A through opening 111 can be provided in the cover 110, and gas in the environment of the gas concentration sensor can pass into the cavity 121 through the through opening.

The resistance sensor elements 122 and the environment thereof form a thermal system that is influenced by the composition of the gas situated in the cavity 121.

The resistance elements 122 situated in the cavity 121 and thus the thermal system can be excited by an input signal. By measuring an output signal of the resistance sensor elements 122 and evaluating same depending on the input signal, it is possible to determine a first parameter of a transfer function describing the thermal system, and a second parameter of the transfer function. By comparing the first parameter and the second parameter, it is possible to check whether the first parameter determined is plausible and, if this is not the case, an error signal can be output.

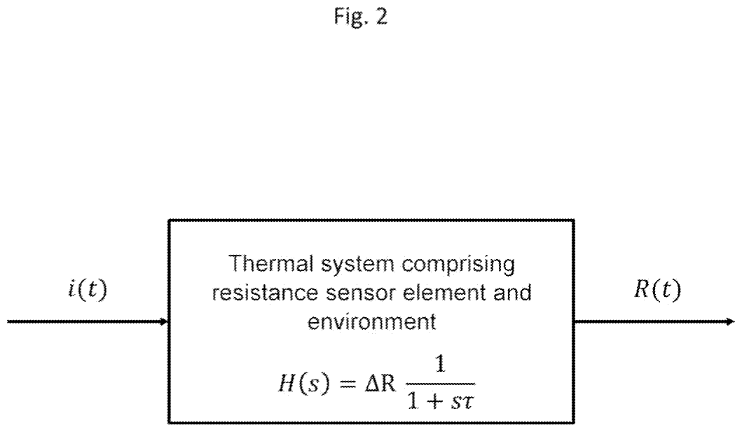

A model of the thermal system is indicated by way of example in FIG. 2. The thermal system can be modeled by a low-pass filter with the transfer function

$$H ( s ) = Δ R \frac{1}{1 + s τ} .$$

In this case, ΔR describes a change in resistance, and τ describes a time constant. As described above, the thermal system can be excited by an input signal i(t). The resistance of the resistance elements can be determined as output signal R(t):

$$R ( t ) = {R}_{0} + Δ R \cdot [ 1 - {e}^{- ( \frac{t - {t}_{0}}{τ} )} ]$$

Both the parameter ΔR and the time constant τ are dependent on the composition of the gas situated in the cavity 121.

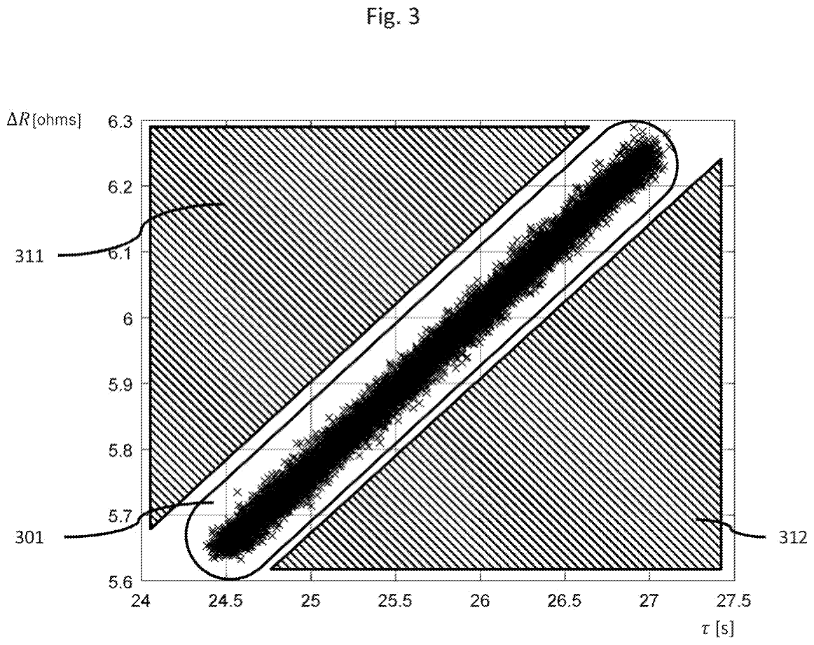

FIG. 3 shows by way of example value pairs for the resistance difference ΔR and the time constant τ for various hydrogen concentrations. In this case, the low values of the resistance difference ΔR and the time constant τ correspond to a hydrogen concentration of approximately 4 percent in the atmosphere under standard conditions, and the high values of the resistance difference ΔR and the time constant τ correspond here to a hydrogen concentration of approximately 0 percent.

As long as the value pairs ΔR and τ are in the range 301, the values can be deemed to be plausible and it is possible to determine the hydrogen concentration from either the parameter ΔR or the parameter τ.

If a value pair determined is outside the range 301, e.g. in the range 311 or in the range 312, the value pair obtained is not plausible. A hydrogen concentration determined with the aid of the parameter ΔR or the parameter τ is thus highly likely to be erroneous. The proposed method thus makes it possible to ascertain whether there is a functional error, and if appropriate to output an error signal.

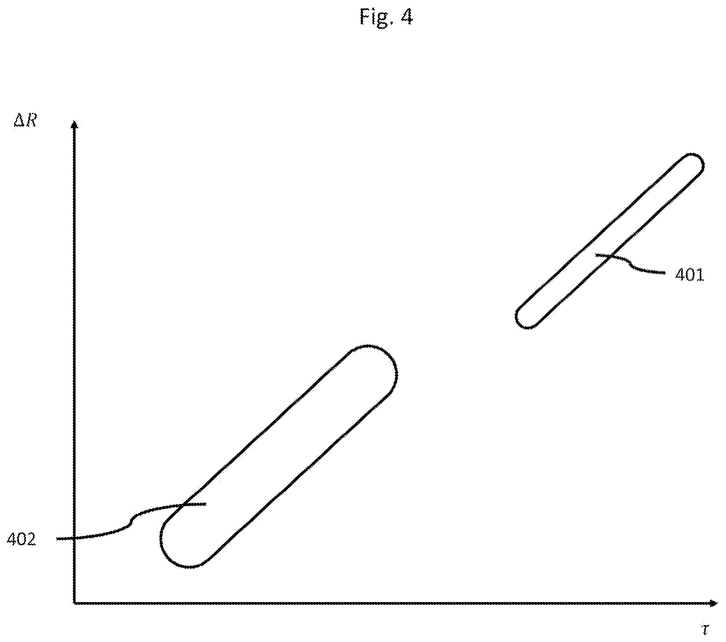

FIG. 4 shows by way of example a parameter value pair range 401 for various H₂ concentrations and parameter value pair ranges 402 for concentrations of other gases in the atmosphere under standard conditions. It is clearly discernible that the parameter value pair ranges 401 and 402 are distinctly separate from one another, and so the H₂ concentration can be determined with high reliability using the method described.

What is thus proposed is a method for determining a gas concentration in a cavity comprising: exciting a resistance sensor element situated in the cavity with an input signal, measuring an output signal of the resistance sensor element, determining a first parameter of a transfer function based on the input signal and the output signal, determining a second parameter of the transfer function based on the input signal and the output signal, checking a plausibility of the first parameter based on the second parameter, and outputting an error signal in the case of lack of plausibility of the first parameter.

The gas concentration sensor 100 can have an integrated circuit 140, which has been fabricated separately from the semiconductor substrate 120 having the resistance sensor elements 122. The integrated circuit 140 can be e.g. an ASIC. The integrated circuit 140 can have an excitation unit for exciting the resistance sensor elements 122 with the input signal, a measuring unit for determining an output signal of the resistance sensor elements, and an evaluation unit for carrying out the proposed method. In principle, it would also be conceivable to realize the excitation unit and/or the measuring unit and/or the evaluation unit in the semiconductor substrate 120.

By way of example, a current signal can be used as input signal. Alternatively, it is conceivable to use a voltage signal. A resistance signal is typically used as output signal. In principle, however, given a current signal as input signal, a voltage signal could be measured as output signal or, given a voltage signal as input signal, a current signal could be measured as output signal.

In example implementations, the thermal system can be modeled by a low-pass filter, in particular a first-order low-pass filter, with a corresponding transfer function. A change in the resistance value can be used as first parameter. It is likewise possible to use a time constant as first parameter. The gas concentration can thus be determined either based on the change in the resistance value or based on the time constant. The proposed gas concentration sensor can be used in particular for determining an H₂ concentration.

### Aspects

Some example implementations are defined by the following aspects:

Aspect 1. A method for determining a gas concentration in a cavity comprising:

exciting a resistance sensor element situated in the cavity with an input signal (i(t)),
measuring an output signal of the resistance sensor element (R(t)),
determining a first parameter (ΔR, τ) of a transfer function

$$( H ( s ) = Δ R \frac{1}{1 + s τ} )$$

based on the input signal (i(t)) and the output signal (R(t)),
determining a second parameter (τ, ΔR) of the transfer function (H(s)) based on the input signal (i(t)) and the output signal (R (t)),
checking a plausibility of the first parameter (ΔR, τ) based on the second parameter (τ, ΔR), and outputting an error signal in the case of lack of plausibility of the first parameter (ΔR, τ).

Aspect 2. The method according to aspect 1,

wherein the input signal (i(t)) is a current signal.

Aspect 3. The method according to either of the preceding aspects,

wherein the input signal is a voltage signal.

Aspect 4. The method according to any of the preceding aspects,

wherein the output signal (R(t)) is a resistance signal.

Aspect 5. The method according to any of the preceding aspects,

wherein the transfer function (H(s)) is a low-pass filter, in particular a first-order low-pass filter.

Aspect 6. The method according to any of the preceding aspects,

wherein the first parameter (ΔR) is a change in a resistance value.

Aspect 7. The method according to any of the preceding aspects 1 to 5,

wherein the first parameter (i) is a time constant.

Aspect 8. The method according to any of the preceding aspects,

wherein the gas concentration (vol.-% H₂) is determined based on the first parameter (ΔR, τ).

Aspect 9. The method according to any of the preceding aspects,

wherein the gas concentration is an H₂ concentration.

Aspect 10. A gas concentration sensor comprising

a cavity for receiving a gas,
a resistance sensor element arranged in the cavity,
an excitation unit for exciting the resistance sensor element with an input signal,
a measuring unit for determining an output signal of the resistance sensor element,
an evaluation unit, wherein the evaluation unit is configured for carrying out the method according to any of aspects 1 to 9.

Although specific example implementations have been illustrated and described in this description, persons having customary knowledge in the art will recognize that a large number of alternative and/or equivalent implementations can be chosen as substitution for the specific example implementations shown and described in this description, without departing from the scope of the implementation disclosed. The intention is for this application to cover all adaptations or variations of the specific example implementations discussed here. Therefore, the intention is for this implementation to be restricted only by the claims and the equivalents of the claims.

1. A method for determining a gas concentration in a cavity, the method comprising:
exciting a resistance sensor element situated in the cavity with an input signal;
measuring an output signal of the resistance sensor element;
determining a first parameter of a transfer function based on the input signal and the output signal;
determining a second parameter of the transfer function based on the input signal and the output signal;
checking a plausibility of the first parameter based on the second parameter; and
outputting an error signal when a lack of plausibility of the first parameter is determined.

2. The method as claimed in claim 1,
wherein the input signal is a current signal.

3. The method as claimed in claim 1,
wherein the input signal is a voltage signal.

4. The method as claimed in claim 1,
wherein the output signal is a resistance signal.

5. The method as claimed in claim 1,
wherein the transfer function is a low-pass filter.

6. The method as claimed in claim 1,
wherein the first parameter is a change in a resistance value.

7. The method as claimed in claim 1,
wherein the first parameter is a time constant.

8. The method as claimed in claim 1, further comprising:
determining the gas concentration based on the first parameter.

9. The method as claimed in claim 1,
wherein the gas concentration is an H₂ concentration.

10. A gas concentration sensor, comprising:
a cavity configured to receive a gas;
a resistance sensor element arranged in the cavity;
an excitation circuit configured to excite the resistance sensor element with an input signal;
a measuring circuit configured to determine an output signal of the resistance sensor element; and
an evaluation circuit, wherein the evaluation circuit is configured to:
determine a first parameter of a transfer function based on the input signal and the output signal;
determine a second parameter of the transfer function based on the input signal and the output signal;
check a plausibility of the first parameter based on the second parameter; and
output an error signal when a lack of plausibility of the first parameter is determined.
